(19) United States
(12) Patent Application Publication
(10) Pub. No.: US 2024/0177397 A1
(43) Pub. Date: May 30, 2024

(54) GENERATION OF DENTAL RENDERINGS FROM MODEL DATA

(71) Applicant: Align Technology, Inc., San Jose, CA (US)

(72) Inventors: Guotu LI, Apex, NC (US); Michael CHANG, Zürich (CH); Christopher Cramer, Durham, NC (US); Michael Austin Brown, Orlando, FL (US); Magdalena Blankenburg, Berlin (DE)

(21) Appl. No.: 18/522,169

(22) Filed: Nov. 28, 2023

Related U.S. Application Data
(60) Provisional application No. 63/428,941, filed on Nov. 30, 2022.

(51) Int. Cl. G06T 15/00; A61C 7/00; A61C 9/00; G16H 30/40

(52) U.S. Cl. CPC G06T 15/005; A61C 7/002; A61C 9/0053; G16H 30/40

(57) ABSTRACT

Methods and systems are described that use a three-dimensional (3D) model of a dental site to generate a panoramic two-dimensional (2D) image of the dental site. In one example, a method includes receiving a 3D model of the dental site generated at least in part from an intraoral scan, generating a surface shaped to substantially surround a dental arch represented by the dental site, computing a surface projection by projecting the dental site onto the surface, and generating the panoramic 2D image from the surface projection.

### RELATED APPLICATION(S)

The present application claims the benefit under 35 U.S.C. § 119(e) of U.S. Provisional Patent Application No. 63/428,941, filed Nov. 30, 2022, which is hereby incorporated by reference herein in its entirety.

### TECHNICAL FIELD

Embodiments of the present disclosure relate to the field of dentistry and, in particular, to the use of three-dimensional (3D) models from intraoral scans to generate two-dimensional (2D) dental arch renderings.

### BACKGROUND

Ionizing radiation has historically been used for imaging teeth, with X-ray bitewing radiograms being the common technique used to provide non-quantitative images of a patient's dentition. However, in addition to the risk of ionizing radiation, such images are typically limited in their ability to show features and may involve a lengthy and expensive procedure to take. Other techniques, such as cone beam computed tomography (CBCT) may provide tomographic images, but still require ionizing radiation.

Specialized 3D scanning tools have also been used to image teeth. Scans from the 3D scanning tools provide topographical data of a patient's dentation that can be used to generate a 3D dental mesh model of the patient's teeth. For restorative dental work such as crowns and bridges, one or more intraoral scans may be generated of a preparation tooth and/or surrounding teeth on a patient's dental arch using an intraoral scanner. Surface representations of the 3D surfaces of teeth have proven extremely useful in the design and fabrication of dental prostheses (e.g., crowns or bridges), and treatment plans.

Two-dimensional (2D) renderings can be readily generated from such 3D models. Traditional rendering approaches often look at a local portion of a patient's jaw, but cannot provide a comprehensive picture of the entire arch. As a result, at least seven images are often required, i.e., right-buccal, right-lingual, anterior-buccal, anterior-lingual, left-buccal, left-lingual, and occlusal views, to have a more complete picture of a jaw. While there are techniques for stitching multiple local tooth arch renderings into a single image, those methods often suffer from unexpected/unwanted distortions. Thus, there is a need for approaches that can minimize or reduce geometric distortions in the rendering process.

### SUMMARY

Multiple example implementations are summarized. Many other implementations are also envisioned.

In a first implementation, a method comprises: receiving a three-dimensional (3D) model of a dental site generated from one or more intraoral scans; generating a projection target shaped to substantially surround an arch represented by the dental site; computing a surface projection by projecting the 3D model of the dental site onto one or more surfaces of the projection target; and generating at least one panoramic 2D image of the dental site from the surface projection.

In a second implementation, a method comprises: receiving a three-dimensional (3D) model of a dental site generated from one or more intraoral scans; generating a plurality of vertices along an arch represented by the dental site; computing a projection target comprising a plurality of surface segments connected to each other in series at the locations of the vertices; scaling the projection target with respect to the arch center located within a central region of the arch such that the projection target substantially surrounds the arch; computing a surface projection by projecting the 3D model of the dental site onto each of the surface segments of the projection target; and generating at least one panoramic 2D image of the dental site from the surface projection.

In a third implementation, a method comprises: receiving a 3D model of a dental site generated from one or more intraoral scans; generating a projection target shaped to substantially surround an arch represented by the dental site; computing a first surface projection by projecting the 3D model of the dental site onto one or more surfaces of the projection target along a buccal direction; computing a second surface projection by projecting the 3D model of the dental site onto one or more surfaces of the projection target along a lingual direction; and generating at least one panoramic 2D image by combining the first surface projection and the second surface projection.

In a fourth implementation, a non-transitory computer readable medium comprises instructions that, when executed by a processing device, cause the processing device to perform the method of any of the preceding implementations.

In a fifth implementation, an intraoral scanning system comprises an intraoral scanner and a computing device operatively connected to the intraoral scanner, wherein the computing device is to perform the method of any of the preceding implementations.

In a sixth implementation, a system comprises a memory and a processing device to execute instructions from the memory to perform the method of any of the preceding implementations.

### BRIEF DESCRIPTION OF THE DRAWINGS

Embodiments of the present disclosure are illustrated by way of example, and not by way of limitation, in the figures of the accompanying drawings.

FIG. 1 illustrates an exemplary system for performing intraoral scanning and/or generating panoramic 2D images of a dental site, in accordance with at least one embodiment.

FIG. 2 illustrates a cylindrical modeling approach for generating a 2D projection of a 3D dentition, in accordance with at least one embodiment.

FIG. 3 illustrates projection of the 3D dentition onto a cylindrical projection surface, in accordance with at least one embodiment.

FIG. 4 is a workflow illustrating generation of an X-ray panoramic simulated image, in accordance with at least one embodiment.

FIG. 5 is a comparison of an actual X-ray image to an X-ray panoramic simulated image, in accordance with at least one embodiment.

FIG. 6A illustrates an arch curve-following modeling approach for generating a 2D projection of a 3D dentition, in accordance with at least one embodiment.

FIG. 6B is a workflow illustrating generation of a panoramic projection from a 3D dentition based on the arch curve-following modeling approach, in accordance with at least one embodiment.

FIG. 7 illustrates a graphical user interface displaying various renderings of a 3D dentition, in accordance with at least one embodiment.

FIG. 8A illustrates a further arch curve-following modeling approach for generating a 2D projection of a 3D dentition, in accordance with at least one embodiment.

FIG. 8B illustrates a 2D buccal rendering of the 3D dentition using the arch curve-following modeling approach, in accordance with at least one embodiment.

FIG. 8C illustrates a 2D lingual rendering of the 3D dentition using the arch curve-following modeling approach, in accordance with at least one embodiment.

FIG. 9A illustrates a polynomial curve modeling approach for generating a 2D projection of a 3D dentition, in accordance with at least one embodiment.

FIG. 9B shows overlays of 2D panoramic renderings onto X-ray images, in accordance with at least one embodiment.

FIG. 10 illustrates a flow diagram for a method of generating a panoramic 2D image, in accordance with at least one embodiment.

FIG. 11 illustrates a flow diagram for a method of generating a panoramic 2D image based on a multi-surface projection target, in accordance with at least one embodiment, in accordance with at least one embodiment.

FIG. 12A illustrates a flow diagram for a method of generating an X-ray panoramic simulated image, in accordance with at least one embodiment.

FIG. 12B illustrates a flow diagram for a method of generating projecting segmentation/classification information from a panoramic 2D image onto a 3D model of a dental site.

FIG. 13 illustrates a block diagram of an example computing device, in accordance with embodiments of the present disclosure.

### DETAILED DESCRIPTION

Described herein are methods and systems using 3D models of a dental site of a patient (e.g., a dentition) to generate panoramic 2D images of the dental site. The 2D images may be used, for example, for inspecting and evaluating the shapes, positions, and orientations of teeth, as well as for identifying and labeling of dental features. Examples of dental features that may be identified and/or labeled include cracks, chips, gum line, worn tooth regions, cavities (also known as caries), emergent profile (e.g., the gum tooth line intersection), an implant gum line, implant edges, scan body edge/curves, margin line of a preparation tooth, and so on. Also described herein are methods and systems for simulating X-ray images from panoramic renderings of 3D models (referred to herein as “X-ray panoramic simulated images”). Also described herein are methods and systems for labeling dental features in panoramic 2D images and assigning labels to corresponding dental features in the 3D model from which the panoramic 2D images are derived. Certain embodiments described herein parameterize the rendering process by projecting the 3D model onto various types of projection targets to reduce or minimize geometric distortions. Certain embodiments further relate to projection targets that closely track the contours of the patient's dental arch. Such embodiments can provide more accurate panoramic renderings with minimal distortion, further facilitating a dentist to conduct visual oral diagnostics and provide patient education.

The embodiments described herein provide a framework for panoramic dental arch renderings (both buccal and lingual views). When combined with the occlusal view of the jaw, dental personnel can have a comprehensive overview of the patient's jaw to facilitate both diagnostics and patient education. Unlike traditional rendering approaches which often require at least seven images (i.e., right-buccal, right-lingual, anterior-buccal, anterior-lingual, left-buccal, left-lingual and occlusal views), the embodiments described herein can reduce the number of renderings used for fully visualizing the patient's dentition down to three, i.e., buccal panoramic, lingual panoramic, and occlusal. Moreover, the panoramic arch rendering provides for easier image labeling for various image-based oral diagnostic modeling processes. Such labeling in 2D can be projected back onto the original 3D model for machine learning purposes, visualization, etc. The panoramic arch rendering also provides an approach to simulating panoramic X-rays, which could potentially reduce or eliminate the need to take actual panoramic X-rays during or after a patient's orthodontic treatment. For example, with a patient's initial panoramic X-ray image and the original 3D scan, the X-ray simulation process can be calibrated, and new X-ray-like images can be rendered even after the patient's teeth have moved due to treatment. This can potentially reduce/eliminate the need for follow-up X-rays during or after a patient's orthodontic treatment, especially when combined with 3D tooth root reconstructions.

Advantages of the embodiments of the present disclosure include, but are not limited to: (1) providing a methodology for rendering panoramic images of a dental arch directly from a 3D scans of a patient's dentition to provide a comprehensive picture of the patient's jaw that facilitates easier oral diagnostics and patient education; (2) facilitating the labeling of various dental features from the panoramic renderings and enabling various image-based machine learning approaches; (3) simulating panoramic X-ray images to potentially reduce or eliminate follow-up X-rays during or after a patient's orthodontic treatment; and (4) utilizing a parametric approach to allow ease of controlling various aspects of final renderings (e.g., the amount of back molar angulation in the panoramic renderings).

Various embodiments are described herein. It should be understood that these various embodiments may be implemented as stand-alone solutions and/or may be combined. Accordingly, references to an embodiment, or one embodiment, may refer to the same embodiment and/or to different embodiments. Some embodiments are discussed herein with reference to intraoral scans and intraoral images. However, it should be understood that embodiments described with reference to intraoral scans also apply to lab scans or model/impression scans. A lab scan or model/impression scan may include one or more images of a dental site or of a model or impression of a dental site, which may or may not include height maps, and which may or may not include color images.

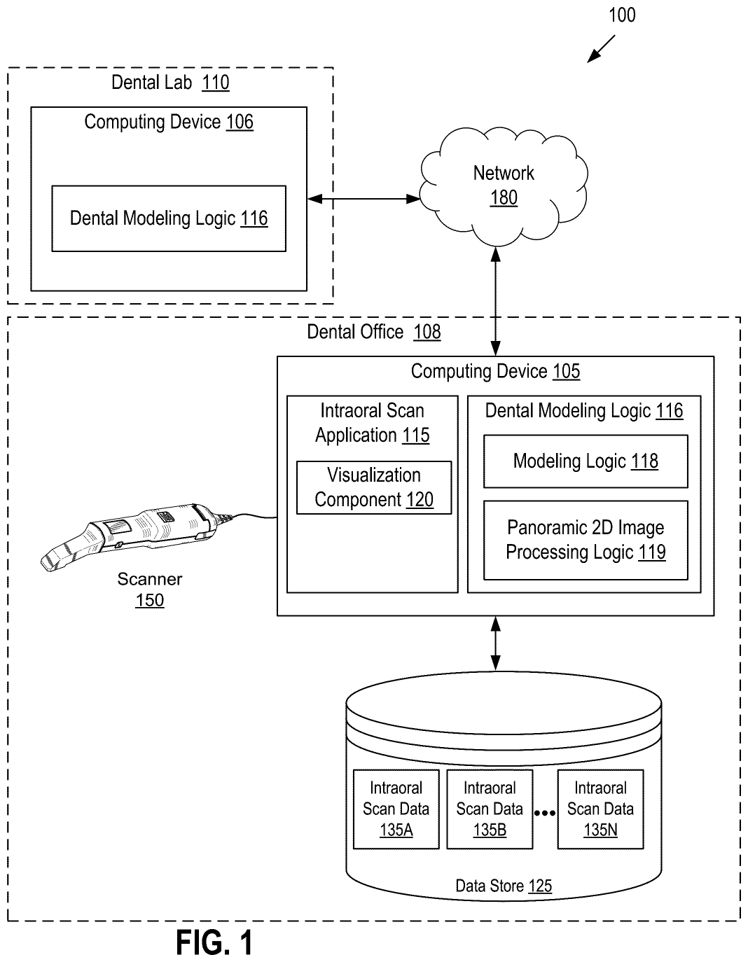

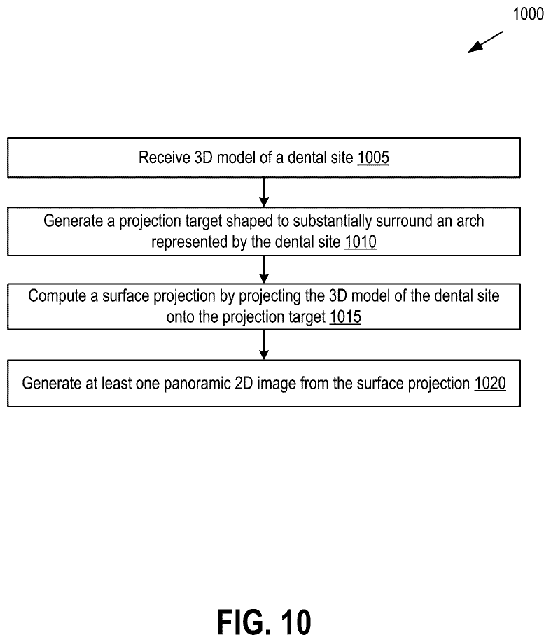

FIG. 1 illustrates an exemplary system 100 for performing intraoral scanning and/or generating panoramic 2D images of a dental site, in accordance with at least one embodiment. In at least one embodiment, one or more components of system 100 carries out one or more operations described below with reference to FIGS. 10-12.

System 100 includes a dental office 108 and a dental lab 110. The dental office 108 and the dental lab 110 each include a computing device 105, 106, where the computing devices 105, 106 may be connected to one another via a network 180. The network 180 may be a local area network (LAN), a public wide area network (WAN) (e.g., the Internet), a private WAN (e.g., an intranet), or a combination thereof.

Computing device 105 may be coupled to an intraoral scanner 150 (also referred to as a scanner) and/or a data store 125. Computing device 106 may also be connected to a data store (not shown). The data stores may be local data stores and/or remote data stores. Computing device 105 and computing device 106 may each include one or more processing devices, memory, secondary storage, one or more input devices (e.g., such as a keyboard, mouse, tablet, and so on), one or more output devices (e.g., a display, a printer, etc.), and/or other hardware components.

In at least one embodiment, scanner 150 is wirelessly connected to computing device 105 via a direct wireless connection. In at least one embodiment, scanner 150 is wirelessly connected to computing device 105 via a wireless network. In at least one embodiment, the wireless network is a Wi-Fi network. In at least one embodiment, the wireless network is a Bluetooth network, a Zigbee network, or some other wireless network. In at least one embodiment, the wireless network is a wireless mesh network, examples of which include a Wi-Fi mesh network, a Zigbee mesh network, and so on. In an example, computing device 105 may be physically connected to one or more wireless access points and/or wireless routers (e.g., Wi-Fi access points/routers). Intraoral scanner 150 may include a wireless module such as a Wi-Fi module, and via the wireless module may join the wireless network via the wireless access point/router.

In embodiments, scanner 150 includes an inertial measurement unit (IMU). The IMU may include an accelerometer, a gyroscope, a magnetometer, a pressure sensor and/or other sensor. For example, scanner 150 may include one or more micro-electromechanical system (MEMS) IMU. The IMU may generate inertial measurement data (referred to herein as movement data or motion data), including acceleration data, rotation data, and so on.

Intraoral scanner 150 may include a probe (e.g., a hand held probe) for optically capturing three-dimensional structures. The intraoral scanner 150 may be used to perform an intraoral scan of a patient's oral cavity, in which a plurality of intraoral scans (also referred to as intraoral images) are generated. An intraoral scan application 115 running on computing device 105 may communicate with the scanner 150 to effectuate the intraoral scanning process. A result of the intraoral scanning may be intraoral scan data 135A, 135B through 135N that may include one or more sets of intraoral scans or intraoral images. Each intraoral scan or image may include a two-dimensional (2D) image that includes depth information (e.g., via a height map of a portion of a dental site) and/or may include a 3D point cloud. In either case, each intraoral scan includes x, y and z information. Some intraoral scans, such as those generated by confocal scanners, include 2D height maps. In at least one embodiment, the intraoral scanner 150 generates numerous discrete (i.e., individual) intraoral scans. Sets of discrete intraoral scans may be merged into a smaller set of blended intraoral scans, where each blended intraoral scan is a combination of multiple discrete intraoral scans. Intraoral scan data 135A-N may optionally include one or more color images (e.g., color 2D images) and/or images generated under particular lighting conditions (e.g., 2D ultraviolet (UV) images, 2D infrared (IR) images, 2D near-IR images, 2D fluorescent images, and so on).

The scanner 150 may transmit the intraoral scan data 135A, 135B through 135N to the computing device 105. Computing device 105 may store the intraoral scan data 135A-135N in data store 125.

According to an example, a user (e.g., a practitioner) may subject a patient to intraoral scanning. In doing so, the user may apply scanner 150 to one or more patient intraoral locations. The scanning may be divided into one or more segments. As an example, the segments may include an upper dental arch segment, a lower dental arch segment, a bite segment, and optionally one or more preparation tooth segments. As another example, the segments may include a lower buccal region of the patient, a lower lingual region of the patient, an upper buccal region of the patient, an upper lingual region of the patient, one or more preparation teeth of the patient (e.g., teeth of the patient to which a dental device such as a crown or other dental prosthetic will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with the scan being directed towards an interface area of the patient's upper and lower teeth). Via such scanner application, the scanner 150 may provide intraoral scan data 135A-N to computing device 105. The intraoral scan data 135A-N may be provided in the form of intraoral scan data sets, each of which may include 3D point clouds, 2D scans/images and/or 3D scans/images of particular teeth and/or regions of an intraoral site. In at least one embodiment, separate data sets are created for the maxillary arch, for the mandibular arch, for a patient bite, and for each preparation tooth. Alternatively, a single large data set is generated (e.g., for a mandibular and/or maxillary arch). Such scans may be provided from the scanner 150 to the computing device 105 in the form of one or more points (e.g., one or more point clouds).

The manner in which the oral cavity of a patient is to be scanned may depend on the procedure to be applied thereto. For example, if an upper or lower denture is to be created, then a full scan of the mandibular or maxillary edentulous arches may be performed. In contrast, if a bridge is to be created, then just a portion of a total arch may be scanned which includes an edentulous region, the neighboring preparation teeth (e.g., abutment teeth) and the opposing arch and dentition. Additionally, the manner in which the oral cavity is to be scanned may depend on a doctor's scanning preferences and/or patient conditions.

By way of non-limiting example, dental procedures may be broadly divided into prosthodontic (restorative) and orthodontic procedures, and then further subdivided into specific forms of these procedures. Additionally, dental procedures may include identification and treatment of gum disease, sleep apnea, and intraoral conditions. The term prosthodontic procedure refers, inter alia, to any procedure involving the oral cavity and directed to the design, manufacture or installation of a dental prosthesis at a dental site within the oral cavity (intraoral site), or a real or virtual model thereof, or directed to the design and preparation of the intraoral site to receive such a prosthesis. A prosthesis may include any restoration such as crowns, veneers, inlays, onlays, implants and bridges, for example, and any other artificial partial or complete denture. The term orthodontic procedure refers, inter alia, to any procedure involving the oral cavity and directed to the design, manufacture or installation of orthodontic elements at a intraoral site within the oral cavity, or a real or virtual model thereof, or directed to the design and preparation of the intraoral site to receive such orthodontic elements. These elements may be appliances including but not limited to brackets and wires, retainers, clear aligners, or functional appliances.

During intraoral scanning, intraoral scan application 115 may register and stitch together two or more intraoral scans (e.g., intraoral scan data 135A and intraoral scan data 135B) generated thus far from the intraoral scan session. In at least one embodiment, performing registration includes capturing 3D data of various points of a surface in multiple scans, and registering the scans by computing transformations between the scans. One or more 3D surfaces may be generated based on the registered and stitched together intraoral scans during the intraoral scanning. The one or more 3D surfaces may be output to a display so that a doctor or technician can view their scan progress thus far.

As each new intraoral scan is captured and registered to previous intraoral scans and/or a 3D surface, the one or more 3D surfaces may be updated, and the updated 3D surface(s) may be output to the display. In embodiments, segmentation is performed on the intraoral scans and/or the 3D surface to segment points and/or patches on the intraoral scans and/or 3D surface into one or more classifications. In at least one embodiment, intraoral scan application 115 classifies points as hard tissue or as soft tissue. The 3D surface may then be displayed using the classification information. For example, hard tissue may be displayed using a first visualization (e.g., an opaque visualization) and soft tissue may be displayed using a second visualization (e.g., a transparent or semi-transparent visualization).

In embodiments, separate 3D surfaces are generated for the upper jaw and the lower jaw. This process may be performed in real time or near-real time to provide an updated view of the captured 3D surfaces during the intraoral scanning process.

When a scan session or a portion of a scan session associated with a particular scanning role or segment (e.g., upper jaw role, lower jaw role, bite role, etc.) is complete (e.g., all scans for an intraoral site or dental site have been captured), intraoral scan application 115 may automatically generate a virtual 3D model of one or more scanned dental sites (e.g., of an upper jaw and a lower jaw). The final 3D model may be a set of 3D points and their connections with each other (i.e., a mesh). In at least one embodiment, the final 3D model is a volumetric 3D model that has both surface and internal features. In embodiments, the 3D model is a volumetric model generated as described in International Patent Application Publication No. WO 2019/147984 A1, entitled “Diagnostic Intraoral Scanning and Tracking,” which is hereby incorporated by reference herein in its entirety.

To generate the virtual 3D model, intraoral scan application 115 may register and stitch together the intraoral scans generated from the intraoral scan session that are associated with a particular scanning role or segment. The registration performed at this stage may be more accurate than the registration performed during the capturing of the intraoral scans, and may take more time to complete than the registration performed during the capturing of the intraoral scans. In at least one embodiment, performing scan registration includes capturing 3D data of various points of a surface in multiple scans, and registering the scans by computing transformations between the scans. The 3D data may be projected into a 3D space of a 3D model to form a portion of the 3D model. The intraoral scans may be integrated into a common reference frame by applying appropriate transformations to points of each registered scan and projecting each scan into the 3D space.

In at least one embodiment, registration is performed for adjacent or overlapping intraoral scans (e.g., each successive frame of an intraoral video). In at least one embodiment, registration is performed using blended scans. Registration algorithms are carried out to register two adjacent or overlapping intraoral scans (e.g., two adjacent blended intraoral scans) and/or to register an intraoral scan with a 3D model, which essentially involves determination of the transformations which align one scan with the other scan and/or with the 3D model. Registration may involve identifying multiple points in each scan (e.g., point clouds) of a scan pair (or of a scan and the 3D model), surface fitting to the points, and using local searches around points to match points of the two scans (or of the scan and the 3D model). For example, intraoral scan application 115 may match points of one scan with the closest points interpolated on the surface of another scan, and iteratively minimize the distance between matched points. Other registration techniques may also be used.

Intraoral scan application 115 may repeat registration for all intraoral scans of a sequence of intraoral scans to obtain transformations for each intraoral scan, to register each intraoral scan with previous intraoral scan(s) and/or with a common reference frame (e.g., with the 3D model). Intraoral scan application 115 may integrate intraoral scans into a single virtual 3D model by applying the appropriate determined transformations to each of the intraoral scans. Each transformation may include rotations about one to three axes and translations within one to three planes.

In many instances, data from one or more intraoral scans does not perfectly correspond to data from one or more other intraoral scans. Accordingly, in embodiments intraoral scan application 115 may process intraoral scans (e.g., which may be blended intraoral scans) to determine which intraoral scans (or which portions of intraoral scans) to use for portions of a 3D model (e.g., for portions representing a particular dental site). Intraoral scan application 115 may use data such as geometric data represented in scans and/or time stamps associated with the intraoral scans to select optimal intraoral scans to use for depicting a dental site or a portion of a dental site. In at least one embodiment, images are input into a machine learning model that has been trained to select and/or grade scans of dental sites. In at least one embodiment, one or more scores are assigned to each scan, where each score may be associated with a particular dental site and indicate a quality of a representation of that dental site in the intraoral scans.

Additionally, or alternatively, intraoral scans may be assigned weights based on scores assigned to those scans (e.g., based on proximity in time to a time stamp of one or more selected 2D images). Assigned weights may be associated with different dental sites. In at least one embodiment, a weight may be assigned to each scan (e.g., to each blended scan) for a dental site (or for multiple dental sites). During model generation, conflicting data from multiple intraoral scans may be combined using a weighted average to depict a dental site. The weights that are applied may be those weights that were assigned based on quality scores for the dental site. For example, processing logic may determine that data for a particular overlapping region from a first set of intraoral scans is superior in quality to data for the particular overlapping region of a second set of intraoral scans. The first intraoral scan data set may then be weighted more heavily than the second intraoral scan data set when averaging the differences between the intraoral scan data sets. For example, the first intraoral scans assigned the higher rating may be assigned a weight of 70% and the second intraoral scans may be assigned a weight of 30%. Thus, when the data is averaged, the merged result will look more like the depiction from the first intraoral scan data set and less like the depiction from the second intraoral scan data set.

In at least one embodiment, images and/or intraoral scans are input into a machine learning model that has been trained to select and/or grade images and/or intraoral scans of dental sites. In at least one embodiment, one or more scores are assigned to each image and/or intraoral scan, where each score may be associated with a particular dental site and indicate a quality of a representation of that dental site in the 2D image and/or intraoral scan. Once a set of images is selected for use in generating a portion of a 3D model/surface that represents a particular dental site (or a portion of a particular dental site), those images/scans and/or portions of those images/scans may be locked. Locked images or portions of locked images that are selected for a dental site may be used exclusively for creation of a particular region of a 3D model (e.g., for creation of the associated tooth in the 3D model).

Intraoral scan application 115 may generate one or more 3D surfaces and/or 3D models from intraoral scans, and may display the 3D surfaces and/or 3D models to a user (e.g., a doctor) via a user interface. The 3D surfaces and/or 3D models can then be checked visually by the doctor. The doctor can virtually manipulate the 3D surfaces and/or 3D models via the user interface with respect to up to six degrees of freedom (i.e., translated and/or rotated with respect to one or more of three mutually orthogonal axes) using suitable user controls (hardware and/or virtual) to enable viewing of the 3D model from any desired direction. The doctor may review (e.g., visually inspect) the generated 3D surface and/or 3D model of an intraoral site and determine whether the 3D surface and/or 3D model is acceptable.

Once a 3D model of a dental site (e.g., of a dental arch or a portion of a dental arch including a preparation tooth) is generated, it may be sent to dental modeling logic 116 for review, analysis and/or updating. Additionally, or alternatively, one or more operations associated with review, analysis and/or updating of the 3D model may be performed by intraoral scan application 115.

Intraoral scan application 115 and/or dental modeling logic 116 may include modeling logic 118 and/or panoramic 2D image processing logic 119. Modeling logic 118 may include logic for generating projection targets onto which a 3D model may be projected. The modeling logic 118 may import the 3D model data to identify various parameters used for generating the projection targets. Such parameters include, but are not limited to, an arch center (which may serve as a projection center for performing projection transformations), a 3D coordinate axis, tooth locations/centers, and arch dimensions. From these parameters, the modeling logic 118 may be able to determine the positions, sizes, and orientations of various projection targets for positioning around the dental arch represented by the 3D model.

In at least one embodiment, the panoramic 2D image processing logic 119 (or “image processing logic”) may utilize one or more models (i.e., projection targets) generated from the modeling logic 118 for generating/deriving panoramic 2D images from the 3D model of the dental site. The image processing logic 119, for example, may generate 2D panoramic images from the 3D model based on the projection center. For example, a radially outward projection onto the projection target may result in a panoramic lingual view of the dentition, and a radially inward projection onto the projection target may result in a panoramic buccal view of the dentition. Image processing logic 119 may also be utilized to generate an X-ray panoramic simulated image from, for example, lingual and buccal 2D panoramic projections. In at least one embodiment, the result of such projection transformations may include not just raw image data, but may also preserve other information related to the 3D model. For example, each pixel of a 2D panoramic image may have associated depth information (e.g., a radial distance from the projection center), density information, 3D surface coordinates, and/or other data. In at least one embodiment, such data may be used in transforming a 2D panoramic image back to a 3D image. In at least one embodiment, such data may be used in identifying overlaps of teeth detectable from the buccal and lingual projections.

In at least one embodiment, a visualization component 120 of the intraoral scan application 115 may be used to visualize the panoramic 2D images for inspection, labeling, patient education, or any other purpose. In at least one embodiment, the visualization component 120 may be utilized to compare panoramic 2D images generated from intraoral scans at various stages of a treatment plan. Such embodiments allow for visualization of tooth movement and shifting. In at least one embodiment, a machine learning model may be trained to detect and automatically label tooth movement and shifting using panoramic 2D images, panoramic X-ray images, and/or intraoral scan data as inputs.

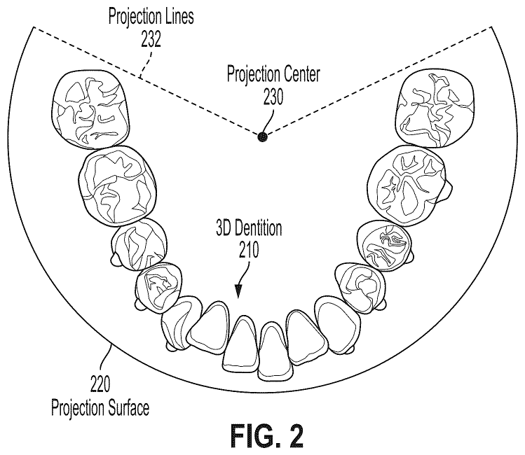

The functionality of the dental modeling logic 116 is now described in greater detail with respect to FIGS. 2-12. Reference is now made to FIG. 2, which illustrates a cylindrical modeling approach for generating a 2D projection of a dental site (e.g., 3D model of a patient's dentition, or “3D dentition” 210), in accordance with at least one embodiment. A top-down view of the 3D dentition 210 is shown with a projection center 230 in a central region of an arch of the 3D dentition 210. A projection target (i.e., projection surface 220) is placed around the dental arch of the 3D dentition 210. As shown, the projection surface 220 is a partial cylinder that surrounds the dental arch. In at least one embodiment, a radius of the projection surface 220 may coincide with the projection center 230. In at least one embodiment, the radius may be selected to surround the dental arch while maintaining a minimum spacing away from the nearest tooth of the 3D dentition 210. In at least one embodiment, the projection center 230 corresponds to the center of the arch. In at least one embodiment, the projection center 230 is selected so that radial projection lines 232 are tangential or nearly tangential to the third molar of the 3D dentition 210 for a given radius of the projection surface 220.

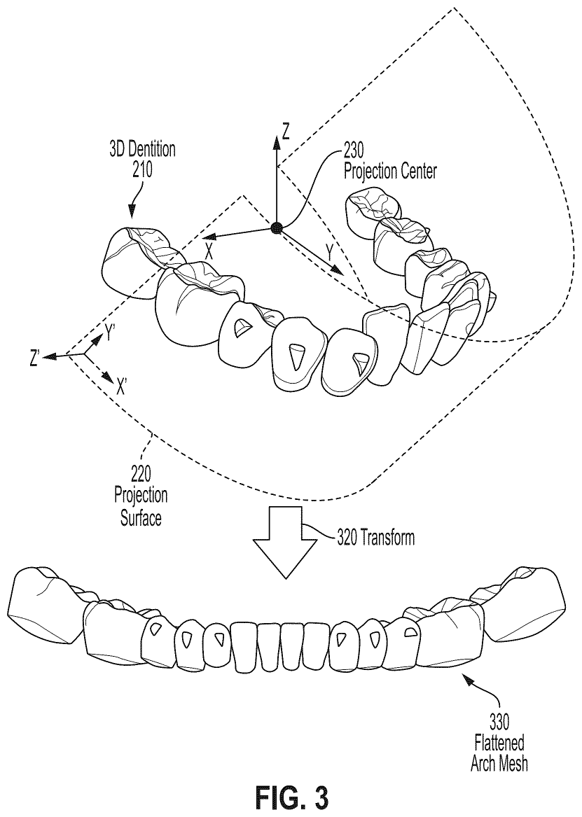

FIG. 3 illustrates projection of the 3D dentition 210 onto the cylindrical projection surface 220, in accordance with at least one embodiment. In at least one embodiment, the 3D dentition 210 is projected onto the cylindrical projection surface 220 and then the 3D model/mesh is flattened to produce a flattened arch mesh 330. The flattened arch mesh 330 can then be rendered using orthographic rendering to generate a panoramic projection. In at least one embodiment, a coordinate system (x-y-z) is based on the original coordinate system associated with the 3D dentition 210, and a coordinate system for the projection surface 220 is defined as x′-y′-z′. In at least one embodiment, the transform 320 is used to transform any coordinate of the 3D dentition 210 to x′-y′-z′ according to the following relationships:

$${x}^{′} = r * {tan}^{- 1} ( \frac{- x}{y} )$$

$${y}^{′} = r * \frac{z}{\sqrt{{x}^{2} + {y}^{2}}}$$

$${z}^{′} = \sqrt{{x}^{2} + {y}^{2}}$$

where r is the distance from the origin of the 3D dentition 210 coordinate system to the projection surface 220. With the above transformation, a “flattened” arch mesh 330 is obtained.

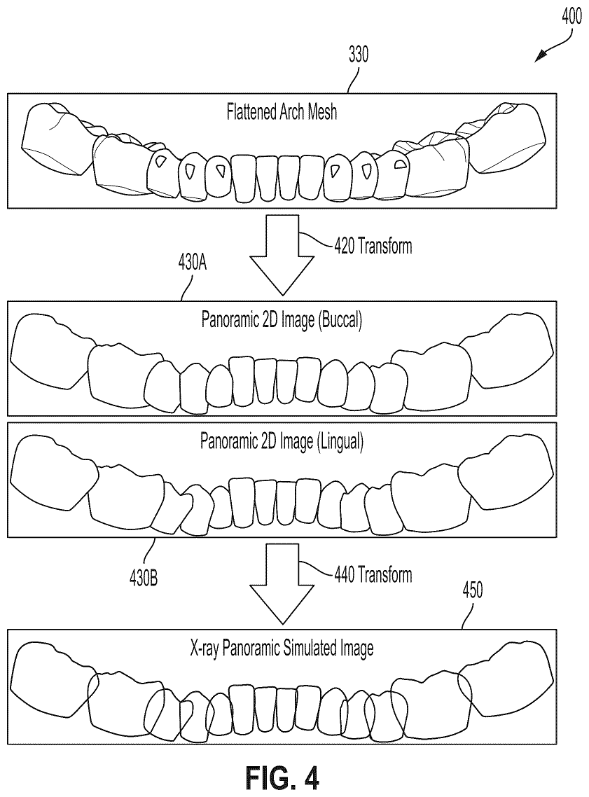

FIG. 4 is a workflow 400 illustrating generation of an X-ray panoramic simulated image 450 (based on circular projection), in accordance with at least one embodiment. In at least one embodiment, orthographic rendering is applied via transformation 420, resulting in panoramic 2D images 430A and/or 430B. In at least one embodiment, applying the transformation results in a buccal image (panoramic 2D image 430A) due to the buccal side of the flattened arch mesh 330 facing the projection surface 220. In at least one embodiment, a lingual rendering (panoramic 2D image 430B) may be obtained, for example, by rotating the flattened arch mesh 330 by 180° about the vertical axis or by flipping the sign of the depth coordinate (z′). In at least one embodiment, the panoramic 2D images 430A and 430B may retain the original color of the 3D dentition 210. In at least one embodiment, the panoramic 2D images 430A and 430B may be recolored. For example, as illustrated in FIG. 4, each tooth is recolored in grayscale using, for example, a gray pixel value of tooth index number multiplied by 5. In at least one embodiment, transform 440 is applied to the panoramic 2D images 430A and 430B to generate an X-ray panoramic simulated image 450, which can be generated by comparing the buccal and lingual renderings of the same jaw, and marking the regions having different color values from each other as a different color (e.g., white) to show tooth overlap that is representative of high density regions of an X-ray image.

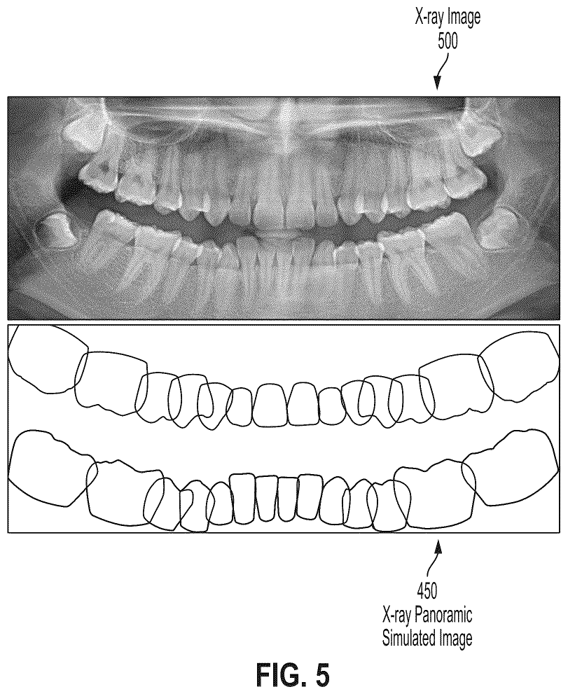

FIG. 5 is a comparison of an actual X-ray image 500 to an X-ray panoramic simulated image 450 for the same patient, in accordance with at least one embodiment. As shown, the simulated rendering of the X-ray panoramic simulated image 450 including the marked/highlighted areas closely resemble the original X-ray image 500, including identification of high-density areas. In at least one embodiment, the simulation process can be calibrated to more closely resemble an X-ray image, for example, by adjusting the location of the projection center and the position and orientation of the projection surface 220. Such calibrations are advantageous, for example, if the patient's jaw was not facing/orthogonal to the X-ray film at the time that the X-ray was captured. In at least one embodiment, these parameters may be iterated through and multiple X-ray panoramic simulated images may be generated in order to identify a best fit simulated image.

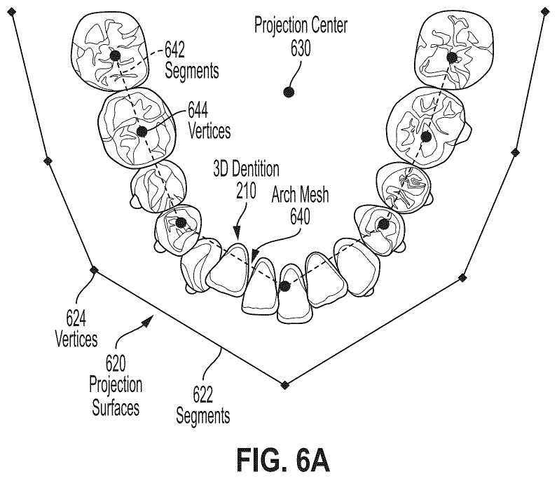

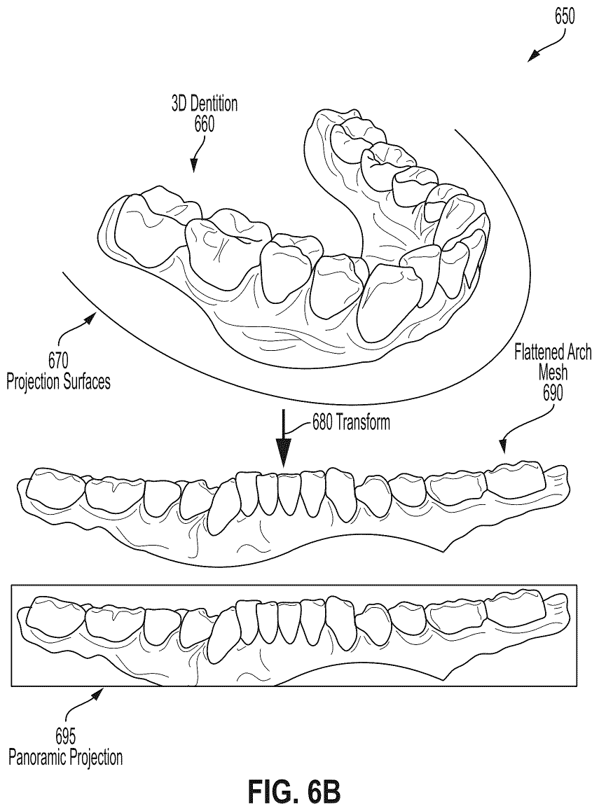

FIG. 6A illustrates an arch curve-following modeling approach for generating a 2D projection of the 3D dentition 210, in accordance with at least one embodiment. In at least one embodiment, a plurality of projection surfaces 620 (e.g., a plurality of connected or continuous projection surfaces 620) are used as the projection target. In at least one embodiment, the dental arch of the 3D dentition is segmented into a plurality of arch segments 642 (e.g., 7 segments as shown) based on the angle span around an arch center. For each of the arch segments 642, a center vertex 644 is calculated. The center vertices 644 are used to connect the segments 642 in a piecewise manner to produce an arch mesh 640. Once generated, the arch mesh 640 is scaled radially outward from the projection center 630 to form the projection surfaces 620 that encompasses the dental arch. In one implementation, a smoothing algorithm is applied to the projection surfaces 620 to produce a more smooth transition between the segments 622 by eliminating/reducing discontinuities caused by the presence of the joints.

FIG. 6B is a workflow 650 illustrating generation of a panoramic projection 695 from a 3D dentition 660 based on the arch curve-following approach, in accordance with at least one embodiment. In at least one embodiment, the projection surfaces 670 can be produced based on (1) a polynomial fitting process, or (2) a similar process as with the projection surfaces 620 utilizing a smoothing algorithm. A transform 680 is applied to the 3D dentition based on the projection surfaces 670 to produce a flattened arch mesh 690. Orthographic rendering is then applied to the flattened arch mesh 690 to generate a final panoramic rendering of the 3D dentition 660 (the panoramic projection 695). As discussed above with respect to FIG. 4, to switch from a buccal view to a lingual view, the sign of the depth coordinate can be switched in the flattened mesh, the vertex order of all triangular mesh faces can be reversed, or the flattened arch mesh 690 can be rotated about its vertical axis prior to applying the orthographic rendering.

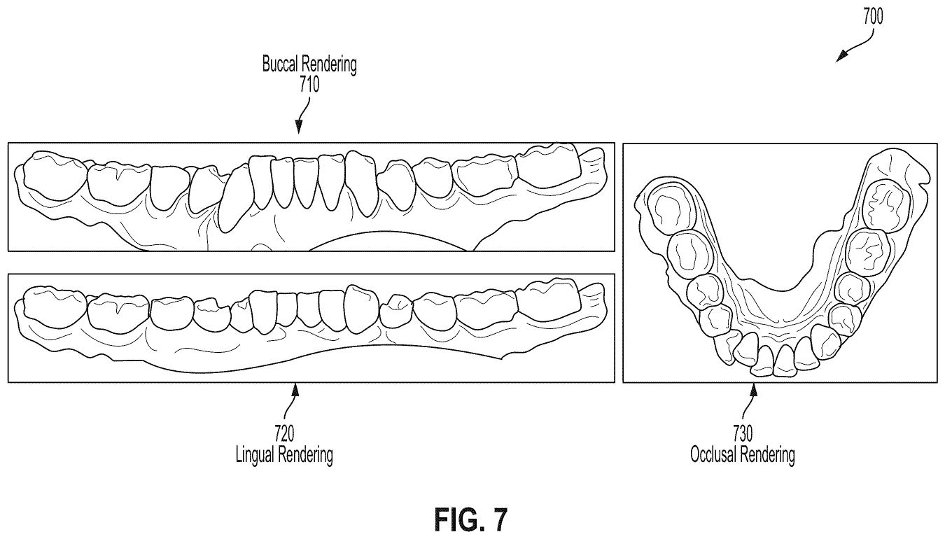

FIG. 7 illustrates a graphical user interface (GUI) 700 displaying various renderings of a 3D dentition, in accordance with at least one embodiment. The GUI 700 may be utilized by dental personnel to display panoramic 2D images of the patient's dentition. As shown, the GUI 700 includes a buccal rendering 710, a lingual rendering 720, and an occlusal rendering 730. The occlusal rendering 730 may be generated, for example, by projecting from a top of the 3D dentition down onto a plane underneath the 3D dentition. In other embodiments, the upper jaw dentition may be shown separately, or the GUI 700 may include renderings of the top and bottom dentitions (e.g., up to 6 total views). In at least one embodiment, the GUI 700 may allow dental personnel to label dental features that are observable in the various renderings. In at least one embodiment, labeling of dental feature in one rendering may cause a similar label to appear in a corresponding location of another rendering.

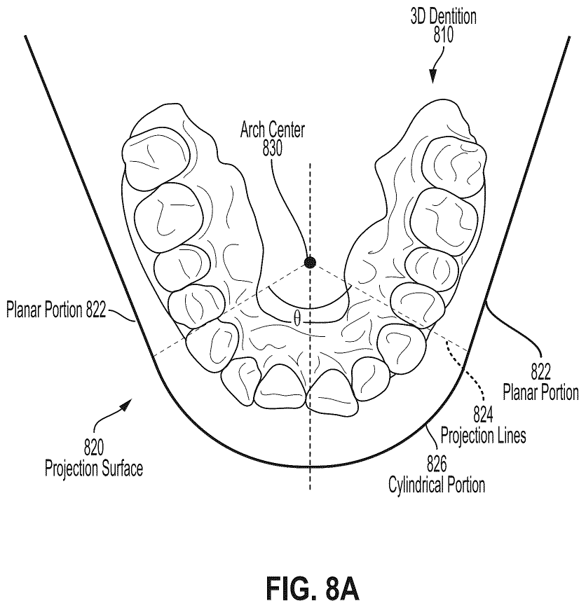

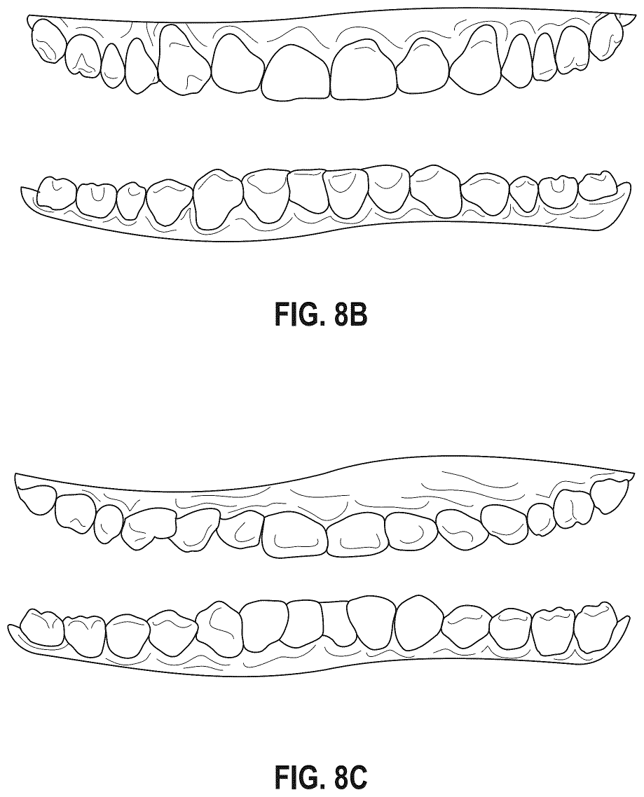

FIG. 8A illustrates an arch curve-following modeling approach utilizing a hybrid projection surface for generating a 2D projection of a 3D dentition 810, in accordance with at least one embodiment. In at least one embodiment, a projection surface 820 is generated from 3 segments joined at their edges, with each section corresponding to a projection sub-surface. In other embodiments, more than 3 segments are utilized. As illustrated, the projection surface 820 is formed from planar portions 822 and a cylindrical portion 826 that connects at its edges to the planar portions 822, resulting in a symmetric shape that substantially surrounds 3D dentition 810. A smooth transition between the planar portions 822 and the cylindrical portion 826 can be utilized to reduce or eliminate discontinuities. In at least one embodiment, the cylindrical portion 826 encompasses a first portion of the 3D dentition 810, and the planar portions 822 extend past the first portion and towards a rear portion of the 3D dentition (left and right back molars). The angle θ corresponds to the angle between two projection lines 824 extending from the projection center 830 to the edges at which the planar portions 822 are connected to the cylindrical portion 826. In at least one embodiment, the angle θ is from about 110° to about 130° (e.g., 120°). In at least one embodiment, the angle θ, the location of the projection center 830, and the orientation and location of the projection surface 820 are used as a tunable parameter to optimize/minimize distortions in the resulting panoramic images. FIGS. 8B and 8C illustrate a 2D buccal rendering and 2D lingual rendering, respectively, of the 3D dentition 810 and of a 3D dentition of the top jaw using the hybrid surface modeling approach, in accordance with at least one embodiment. In at least one embodiment, the renderings may be presented in a GUI for inspection, evaluation, and labeling (as described above with respect to FIG. 7).

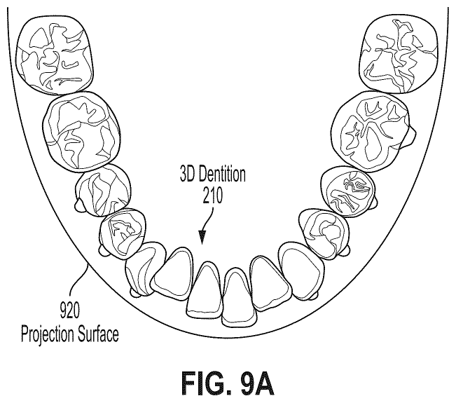

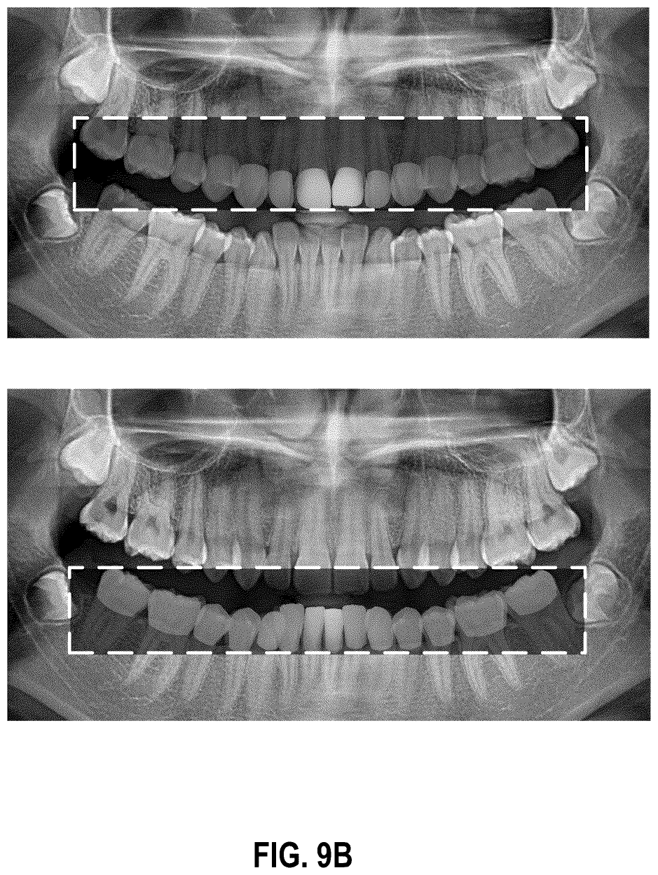

Various other embodiments relate to different geometries for the projection targets. A more generalized arch-curve following approach is now described to further minimize potential distortions in the rendering process. FIG. 9A illustrates a polynomial arch curve modeling approach for generating a 2D projection of the 3D dentition 210, in accordance with at least one embodiment. As shown, a parabolic projection surface 920 surrounds the dental arch of the 3D dentition 210. The parabolic projection surface 920 is an illustrative example of the polynomial curve modeling approach, and it is contemplated that higher-order polynomials may be used as would be appreciated by those of ordinary skill in the art. Such embodiments may be preferable compared to, for example, the cylindrically-shaped projection targets (e.g., the projection surface 220) as parabolically-shaped surfaces may more closely track the patient's dental arch. FIG. 9B shows overlays of 2D panoramic renderings onto X-ray images for the upper and lower arches of a patient, demonstrating the accuracy of the polynomial arch curve modeling approach.

In practice, while all panoramic X-ray imaging systems try to follow patient's arch curve as much as possible during the imaging process, the actual projection trajectory can vary among different panoramic X-ray imaging systems, depending on the underlying design and manufacturing of the imaging systems. Further, a patient's relative positioning to the panoramic X-ray imaging system could also affect the resulting X-ray images. To improve simulation of X-ray images, certain embodiments parameterize both the projection trajectory and the relative jaw positioning to more accurately simulate the panoramic images.

FIGS. 10-12 illustrate methods related to generation of panoramic 2D images from 3D models of dental sites, for which the 3D model is generated from one or more intraoral scans. The methods may be performed by a processing logic that may comprise hardware (e.g., circuitry, dedicated logic, programmable logic, microcode, etc.), software (e.g., instructions run on a processing device to perform hardware simulation), or a combination thereof. In at least one embodiment, at least some operations of the methods are performed by a computing device executing dental modeling application, such as dental modeling logic 116 of FIG. 1. The dental modeling 116 may be, for example, a component of an intraoral scanning apparatus that includes a handheld intraoral scanner and a computing device operatively coupled (e.g., via a wired or wireless connection) to the handheld intraoral scanner. Alternatively, or additionally, the dental modeling application may execute on a computing device at a dentist office or dental lab.

For simplicity of explanation, the methods are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

References now made to FIG. 10, which illustrates a flow diagram for a method 1000 of generating a panoramic 2D image, in accordance with at least one embodiment. At block 1005, a computing device (e.g., the computing device 105 of the dental office 108 or dental lab 110) receives a 3D model of a dental site. In at least one embodiment, the 3D model is generated from one or more intraoral scans. For example, the intraoral scan may be performed by a scanner (e.g., the scanner 150), which generates one or more intraoral scan data sets. In at least one embodiment, the intraoral scan data set may include 3D point clouds, 2D images, and/or 3D images of particular teeth and/or regions of the dental site. The intraoral scan data sets may be processed (e.g., via an intraoral scan application 115 implementing dental modeling logic 116) to produce a 3D model of the dental site, such as a 3D dentition for the lower jaw, the upper jaw, or both, of the patient (e.g., any of the 3D dentitions 210, 660, or 810).

At block 1010, the computing device (e.g., implementing the modeling logic 118) generates a projection target shaped to substantially surround an arch represented by the dental site. In at least one embodiment, the projection target is a cylindrically-shaped surface (e.g., the projection surface 220) that substantially surrounds the arch. In at least one embodiment, the projection target comprises a polynomial curve-shaped curve, such as a parabolically-shaped surface (e.g., the projection surface 620), that substantially surrounds the arch.

In at least one embodiment, the projection target is a hybrid surface (e.g., the projection surface 920) formed from a cylindrically-shaped surface (e.g., the cylindrical portion 926), a first and second planar surfaces (e.g., the planar portions 922) that extend from edges of the cylindrically-shaped surface. In at least one embodiment, the cylindrically-shaped surface, the first planar surface, and the second planar surface collectively define a continuous surface that substantially surrounds the arch. In at least one embodiment, an angle between the first planar surface and the second planar surface is from about 110° to about 130° (e.g., 120°).

At block 1015, the computing device (e.g., implementing the panoramic 2D image processing logic 119) computes a surface projection by projecting the 3D model of the dental site onto one or more surfaces of the projection target. In at least one embodiment, the surface projection is computed based on a projection path surrounding the arch.

At block 1020, the computing device (e.g., implementing the panoramic 2D image processing logic 119) generates at least one panoramic two-dimensional (2D) image from the surface projection. In at least one embodiment, at least one panoramic 2D image is generated by orthographic rendering of a flattened mesh generated by projecting the 3D model along a projection path surrounding the arch (e.g., applying any of transforms 420 or 780).

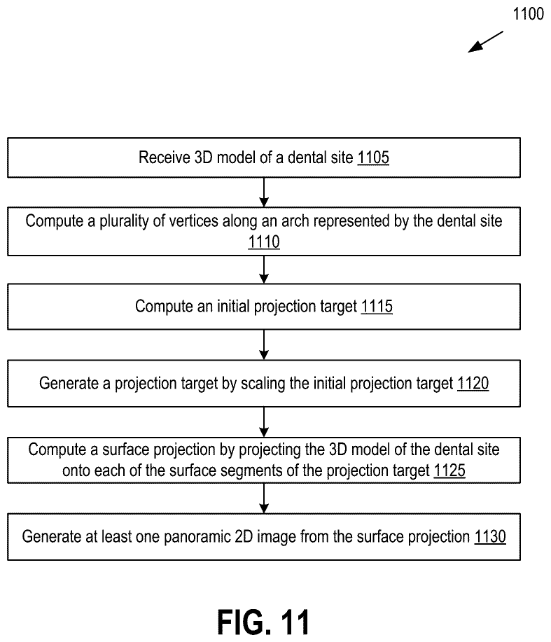

FIG. 11 illustrates a flow diagram for a method 1100 of generating a panoramic 2D image based on a multi-surface projection target, in accordance with at least one embodiment. For example, the method 1100 may follow the workflow described with respect to FIGS. 7A and 7B. At block 1105, a computing device (e.g., the computing device 105 of the dental office 108 or dental lab 110) receives a 3D model of a dental site. In at least one embodiment, the 3D model of the dental site may include a 3D dentition for the lower jaw, the upper jaw, or both, of the patient (e.g., any of the 3D dentitions 210, 660, or 810).

At block 1110, a plurality of vertices (e.g., center vertices 644) are computed along an arch represented by the dental site (e.g., the 3D dentition 210). In at least one embodiment, one or more of the plurality of vertices is positioned at a tooth center. In at least one embodiment, the number of vertices is greater than 5 (e.g., 10, 50, or greater).

At block 1115, an initial projection target is computed (e.g., the arch mesh 640). In at least one embodiment, the initial projection target is formed from a plurality of surface segments (e.g., segments 742) connected to each other in series at the location of the vertices.

At block 1120, a projection target (e.g., the projection surfaces 620) is generated by scaling the initial projection target with respect to the arch center located within a central region of the arch such that the projection target substantially surrounds the arch. The resulting projection target includes a plurality of segments (e.g., segments 622).

At block 1125, a surface projection is computed by projecting the 3D model of the dental site onto each of the surface segments of the projection target. In at least one embodiment, a smoothing algorithm is applied to the projection target to reduce potential discontinuities in the final renderings, thus improving the rendering quality. At block 1130, at least one panoramic two-dimensional (2D) image is generated from the surface projection.

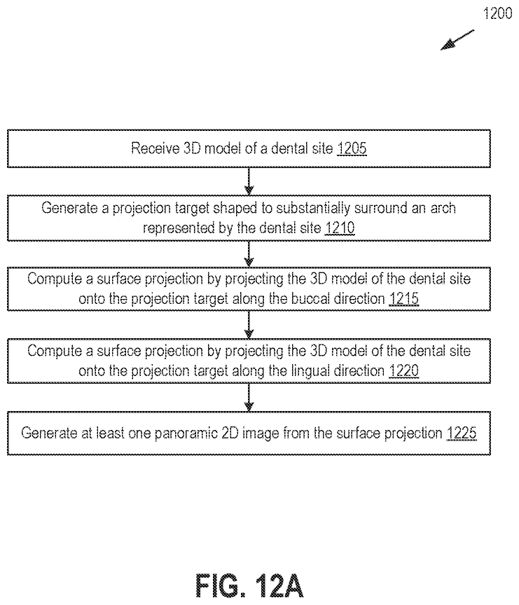

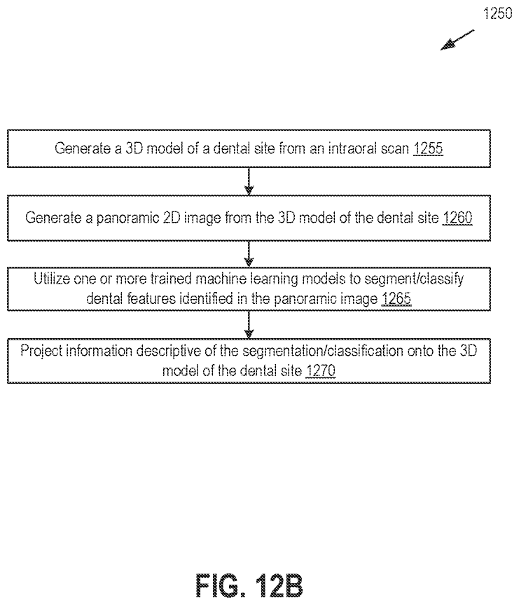

FIG. 12A illustrates a flow diagram for a method 1200 of generating an X-ray panoramic simulated image, in accordance with at least one embodiment. For example, the method 1200 may follow the workflow 400 described with respect to FIG. 4. At block 1205, a computing device (e.g., the computing device 105 of the dental office 108 or dental lab 110) receives a 3D model of a dental site. In at least one embodiment, the 3D model of the dental site may include a 3D dentition for the lower jaw, the upper jaw, or both, of the patient (e.g., any of the 3D dentitions 210, 660, or 810).

At block 1210, a projection target is generated. The projection target may be shaped to substantially surround an arch represented by the dental site. The projection target may correspond to any of those described above with respect to the methods 1000 and 1100.

At block 1215, a first surface projection is computed by projecting the 3D model of the dental site onto one or more surfaces of the projection target along the buccal direction (e.g., based on the transform 420). For example, the projection may be computed by utilizing the mathematical operation described above with respect to FIG. 4 to transform the coordinates of a 3D dentition.

At block 1220, a second surface projection is computed by projecting the 3D model of the dental site onto one or more surfaces of the projection target along the lingual direction (e.g., based on the transform 420). In at least one embodiment, the projection along the lingual direction is performed by flipping the sign of the depth coordinate before or after applying the second surface projection, the vertex order of all mesh faces of the 3D model can be reversed, or the second surface projection can be rotated about its vertical axis.

At block 1225, at least one panoramic 2D image is generated by combining the first surface projection and the second surface projection (e.g., applying transform 440). In at least one embodiment, the resulting panoramic 2D image corresponds to an X-ray panoramic simulated image (e.g., X-ray panoramic simulated image 450). In at least one embodiment, generating the panoramic 2D image includes marking regions of a panoramic 2D image corresponding to overlapping regions of the 3D model identified from the first and second surface projections.

The following embodiments relate to any of the methods 1000, 1100, or 1200. In at least one embodiment, the dental site corresponds to a single jaw. In such embodiments, a first panoramic 2D image can corresponds to a buccal rendering, and a second panoramic 2D image can corresponds to a lingual rendering. The buccal and lingual renderings of the jaw can be displayed, for example, in a GUI individually, together, with an occlusal rendering of the dental site, or with similar renderings for the opposite jaw. In at least one embodiment, the occlusal rendering is generated by projecting the 3D model of the dental site onto a flat surface from the occlusal side of the dental site.

In at least one embodiment, the computing device may generate for display a panoramic 2D image for labeling one or more dental features in the image. Each labeled dental feature has an associated position within the panoramic 2D image. In at least one embodiment, the computing device determines a corresponding location in the 3D model from which the panoramic 2D image was generated and assigns a label for the dental feature to the corresponding location. In at least one embodiment, the 3D model, when displayed, will include the one or more labels. In at least one embodiment, the labeling may be performed, for example, in response to a user input to directly label the dental feature.

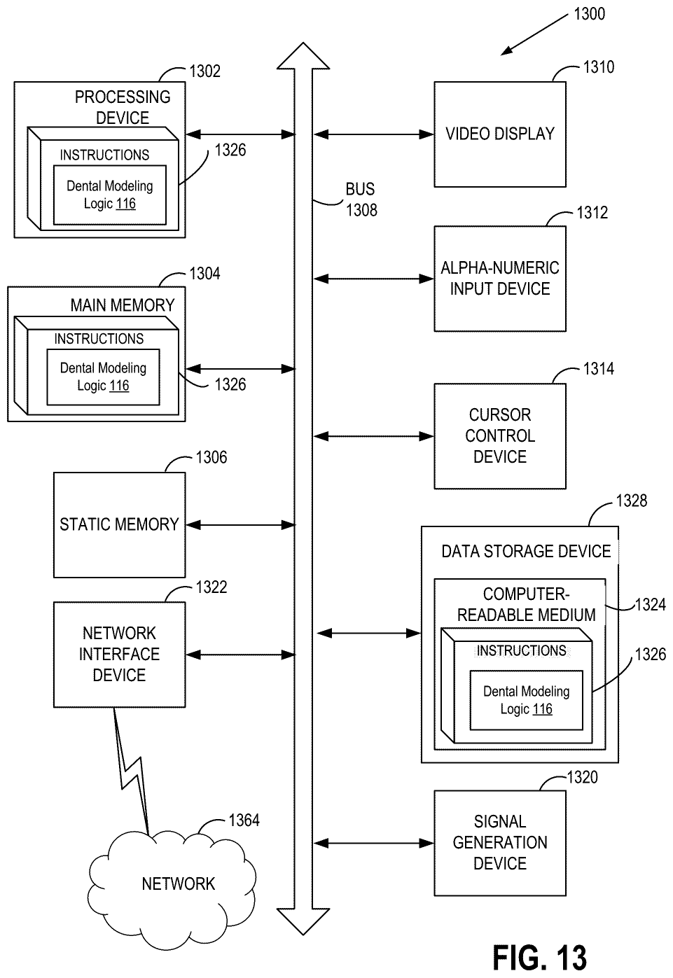

In at least one embodiment, the labeling may be performed using a trained machine learning model. For example, the trained machine learning model can be trained to identify and label dental features in panoramic 2D images, 3D dentitions, or both. In at least one embodiment, one or more workflows may be utilized to implement model training in accordance with embodiments of the present disclosure. In various embodiments, the model training workflow may be performed at a server which may or may not include an intraoral scan application. The model training workflow and the model application workflow may be performed by processing logic executed by a processor of a computing device. One or more of these workflows may be implemented, for example, by one or more machine learning modules implemented in an intraoral scan application 115, by dental modeling logic 116, or other software and/or firmware executing on a processing device of computing device 1300 shown and described in FIG. 13.

The model training workflow is to train one or more machine learning models (e.g., deep learning models) to perform one or more classifying, segmenting, detection, recognition, prediction, etc. tasks for intraoral scan data (e.g., 3D intraoral scans, height maps, 2D color images, 2D NIRI images, 2D fluorescent images, etc.) and/or 3D surfaces generated based on intraoral scan data. The model application workflow is to apply the one or more trained machine learning models to perform the classifying, segmenting, detection, recognition, prediction, etc. tasks for intraoral scan data (e.g., 3D scans, height maps, 2D color images, NIRI images, etc.) and/or 3D surfaces generated based on intraoral scan data. One or more of the machine learning models may receive and process 3D data (e.g., 3D point clouds, 3D surfaces, portions of 3D models, etc.). One or more of the machine learning models may receive and process 2D data (e.g., 2D panoramic images, height maps, projections of 3D surfaces onto planes, etc.).

Many different machine learning outputs are described herein. Particular numbers and arrangements of machine learning models are described and shown. However, it should be understood that the number and type of machine learning models that are used and the arrangement of such machine learning models can be modified to achieve the same or similar end results. Accordingly, the arrangements of machine learning models that are described and shown are merely examples and should not be construed as limiting.

In various embodiments, one or more machine learning models are trained to perform one or more of the below tasks. Each task may be performed by a separate machine learning model. Alternatively, a single machine learning model may perform each of the tasks or a subset of the tasks. Additionally, or alternatively, different machine learning models may be trained to perform different combinations of the tasks. In an example, one or a few machine learning models may be trained, where the trained ML model is a single shared neural network that has multiple shared layers and multiple higher level distinct output layers, where each of the output layers outputs a different prediction, classification, identification, etc. The tasks that the one or more trained machine learning models may be trained to perform are as follows:
I) Canonical position determination—this can include determining canonical position and/or orientation of a 3D surface or of objects in an intraoral scan, or determining canonical positions of objections in a 2D image.
II) Scan/2D image assessment—this can include determining quality metric values associated with intraoral scans, 2D images and/or regions of 3D surfaces. This can include assigning a quality value to individual scans, 3D surfaces, portions of 3D surface, 3D models, portions of 3D models, 2D images, portions of 2D images, etc.
III) Moving tissue (excess tissue) identification/removal—this can include performing pixel-level identification/classification of moving tissue (e.g., tongue, finger, lips, etc.) from intraoral scans and/or 2D images and optionally removing such moving tissue from intraoral scans, 2D images and/or 3D surfaces. Moving tissue identification and removal is described in U.S. Patent Application Publication No. 2020/0349698 A1, entitled “Excess Material Removal Using Machine Learning,” which is hereby incorporated by reference herein in its entirety.
IV) Dental features in 2D or 3D images—this can include performing point-level or pixel-level classification of 3D models and/or 2D images to classify points/pixels as being part of dental features. This can include performing segmentation of 3D surfaces and/or 2D images. Points/pixels may be classified into two or more classes. A minimum classification taxonomy may include a dental feature class and a not dental feature class. In other examples, further dental classes may be identified, such as a hard tissue or tooth class, a soft tissue or gingiva class, and a margin line class.

One type of machine learning model that may be used to perform some or all of the above asks is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g., classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, lips, gums, etc.); and the fourth layer may recognize a scanning role. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

In at least one embodiment, a graph neural network (GNN) architecture is used that operates on three-dimensional data. Unlike a traditional neural network that operates on two-dimensional data, the GNN may receive three-dimensional data (e.g., 3D surfaces) as inputs, and may output predictions, estimates, classifications, etc. based on the three-dimensional data.

In at least one embodiment, a U-net architecture is used for one or more machine learning model. A U-net is a type of deep neural network that combines an encoder and decoder together, with appropriate concatenations between them, to capture both local and global features. The encoder is a series of convolutional layers that increase the number of channels while reducing the height and width when processing from inputs to outputs, while the decoder increases the height and width and reduces the number of channels. Layers from the encoder with the same image height and width may be concatenated with outputs from the decoder. Any or all of the convolutional layers from encoder and decoder may use traditional or depth-wise separable convolutions.

In at least one embodiment, one or more machine learning model is a recurrent neural network (RNN). An RNN is a type of neural network that includes a memory to enable the neural network to capture temporal dependencies. An RNN is able to learn input-output mappings that depend on both a current input and past inputs. The RNN will address past and future scans and make predictions based on this continuous scanning information. RNNs may be trained using a training dataset to generate a fixed number of outputs (e.g., to classify time varying data such as video data as belonging to a fixed number of classes). One type of RNN that may be used is a long short term memory (LSTM) neural network.

A common architecture for such tasks is LSTM (Long Short Term Memory). Unfortunately, LSTM is not well suited for images since it does not capture spatial information as well as convolutional networks do. For this purpose, one can utilize ConvLSTM—a variant of LSTM containing a convolution operation inside the LSTM cell. ConvLSTM is a variant of LSTM (Long Short-Term Memory) containing a convolution operation inside the LSTM cell. ConvLSTM replaces matrix multiplication with a convolution operation at each gate in the LSTM cell. By doing so, it captures underlying spatial features by convolution operations in multiple-dimensional data. The main difference between ConvLSTM and LSTM is the number of input dimensions. As LSTM input data is one-dimensional, it is not suitable for spatial sequence data such as video, satellite, radar image data set. ConvLSTM is designed for 3-D data as its input. In at least one embodiment, a CNN-LSTM machine learning model is used. A CNN-LSTM is an integration of a CNN (Convolutional layers) with an LSTM. First, the CNN part of the model processes the data and a one-dimensional result feeds an LSTM model.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

For the model training workflow, a training dataset containing hundreds, thousands, tens of thousands, hundreds of thousands or more intraoral scans, 2D panoramic images and/or 3D models should be used. In embodiments, up to millions of cases of patient dentition that may have underwent a prosthodontic procedure and/or an orthodontic procedure may be available for forming a training dataset, where each case may include various labels of one or more types of useful information. Each case may include, for example, data showing a 3D model, intraoral scans, height maps, color images, NIRI images, etc. of one or more dental sites, data showing pixel-level segmentation of the data (e.g., 3D model, intraoral scans, height maps, color images, NIRI images, etc.) into various dental classes (e.g., tooth, gingiva, moving tissue, saliva, blood, etc.), data showing one or more assigned scan quality metric values for the data, movement data associated with the 3D scans, and so on. This data may be processed to generate one or multiple training datasets for training of one or more machine learning models. The training datasets may include, for example, a first training dataset of 2D panoramic images with labeled dental features (e.g., cracks, chips, gum line, worn tooth regions, caries, emergent profile, implant gum lines, implant edges, scan body edge/curves, etc.) and a second data set of 3D dentitions with labeled dental features. The machine learning models may be trained, for example, to detect blood/saliva, to detect moving tissue, perform segmentation of 2D images and/or 3D models of dental sites (e.g., to segment such images/3D surfaces into one or more dental classes), and so on.

To effectuate training, processing logic inputs the training dataset(s) into one or more untrained machine learning models. Prior to inputting a first input into a machine learning model, the machine learning model may be initialized. Processing logic trains the untrained machine learning model(s) based on the training dataset(s) to generate one or more trained machine learning models that perform various operations as set forth above.

Training may be performed by inputting one or more of the panoramic 2D images, scans or 3D surfaces (or data from the images, scans or 3D surfaces) into the machine learning model one at a time. Each input may include data from a panoramic 2D image, intraoral scan or 3D surface in a training data item from the training dataset. The training data item may include, for example, a height map, 3D point cloud or 2D image and an associated probability map, which may be input into the machine learning model.

The machine learning model processes the input to generate an output. An artificial neural network includes an input layer that consists of values in a data point (e.g., intensity values and/or height values of pixels in a height map). The next layer is called a hidden layer, and nodes at the hidden layer each receive one or more of the input values. Each node contains parameters (e.g., weights) to apply to the input values. Each node therefore essentially inputs the input values into a multivariate function (e.g., a non-linear mathematical transformation) to produce an output value. A next layer may be another hidden layer or an output layer. In either case, the nodes at the next layer receive the output values from the nodes at the previous layer, and each node applies weights to those values and then generates its own output value. This may be performed at each layer. A final layer is the output layer, where there is one node for each class, prediction and/or output that the machine learning model can produce. For example, for an artificial neural network being trained to determine a dental feature in a 2D panoramic image or a 3D dentition (e.g., represented by a mesh or point cloud).

Processing logic may then compare the determined dental feature to a labeled dental feature of the panoramic 2D image or 3D point cloud. Processing logic determines an error (i.e., a positioning error) based on the differences between the output dental feature and the known correct dental feature. Processing logic adjusts weights of one or more nodes in the machine learning model based on the error. An error term or delta may be determined for each node in the artificial neural network. Based on this error, the artificial neural network adjusts one or more of its parameters for one or more of its nodes (the weights for one or more inputs of a node). Parameters may be updated in a back propagation manner, such that nodes at a highest layer are updated first, followed by nodes at a next layer, and so on. An artificial neural network contains multiple layers of “neurons,” where each layer receives as input values from neurons at a previous layer. The parameters for each neuron include weights associated with the values that are received from each of the neurons at a previous layer. Accordingly, adjusting the parameters may include adjusting the weights assigned to each of the inputs for one or more neurons at one or more layers in the artificial neural network.

Once the model parameters have been optimized, model validation may be performed to determine whether the model has improved and to determine a current accuracy of the deep learning model. After one or more rounds of training, processing logic may determine whether a stopping criterion has been met. A stopping criterion may be a target level of accuracy, a target number of processed images from the training dataset, a target amount of change to parameters over one or more previous data points, a combination thereof and/or other criteria. In at least one embodiment, the stopping criteria is met when at least a minimum number of data points have been processed and at least a threshold accuracy is achieved. The threshold accuracy may be, for example, 70%, 80% or 90% accuracy. In at least one embodiment, the stopping criteria is met if accuracy of the machine learning model has stopped improving. If the stopping criterion has not been met, further training is performed. If the stopping criterion has been met, training may be complete. Once the machine learning model is trained, a reserved portion of the training dataset may be used to test the model.

Once one or more trained ML models are generated, they may be stored in the data store 125, and may be added to the intraoral scan application 115 and/or utilized by the dental modeling logic 116. Intraoral scan application 115 and/or dental modeling logic 116 may then use the one or more trained ML models as well as additional processing logic to identify dental features in panoramic 2D images. The trained machine learning models may be trained to perform one or more tasks in embodiments. In at least one embodiment, the trained machine learning models are trained to perform one or more of the tasks set forth in U.S. Patent Application Publication No. 2021/0059796 A1, entitled “Automated Detection, Generation, And/or Correction of Dental Features in Digital Models,” which is hereby incorporated by reference herein in its entirety. In at least one embodiment, the trained machine learning models are trained to perform one or more of the tasks set forth in U.S. Patent Application Publication No. 2021/0321872 A1, entitled “Smart Scanning for Intraoral Scans,” which is hereby incorporated by reference herein in its entirety. In at least one embodiment, the trained machine learning models are trained to perform one or more of the tasks set forth in U.S. Patent Application Publication No. 2022/0202295 A1, entitled “Dental Diagnostics Hub,” which is hereby incorporated by reference herein in its entirety.

In at least one embodiment, model application workflow includes a first trained model and a second trained model. First and second trained models may each be trained to perform segmentation of an input and identify a dental feature therefrom, but may be trained to operate on different types of data. For example, first trained model may be trained to operate on 3D data, and second trained model may be trained to operate on panoramic 2D images. In at least one embodiment, a single trained machine learning model is used for analyzing multiple types of data.

According to one embodiment, an intraoral scanner generates a sequence of intraoral scans and 2D images. A 3D surface generator may perform registration between intraoral scans to stitch the intraoral scans together and generate a 3D surface/model from the intraoral scans. Additionally, 2D intraoral images (e.g., color 2D images and/or NIRI 2D images) may be generated. Additionally, as intraoral scans and 2D images are generated, motion data may be generated by an IMU of the intraoral scanner and/or based on analysis of the intraoral scans and/or 2D intraoral images.

Data from the 3D model/surface may be input into first trained model, which outputs a first dental feature. The first dental feature may be output as a probability map or mask in at least one embodiment, where each point has an assigned probability of being part of a dental feature and/or an assigned probability of not being part of a dental feature. Similarly, for each panoramic 2D image, data from the panoramic 2D image is input into second trained model which outputs dental feature. The dental feature(s) may each be output as a probability map or mask in at least one embodiment, where each pixel of the input 2D image has an assigned probability of being a dental feature and/or an assigned probability of not being a dental feature.

In at least one embodiment, the machine learning model is additionally trained to identify teeth, gums and/or excess material. In at least one embodiment, the machine learning model is further trained to determine one or more specific tooth numbers and/or to identify a specific indication (or indications) for an input image. Accordingly, a single machine learning model may be trained to identify dental features and also to identify teeth generally, identify different specific tooth numbers, identify gums and/or identify other features (e.g., margin lines, etc.). In an alternative embodiment, a separate machine learning model is trained for each specific tooth number and for each specific indication. Accordingly, the tooth number and/or indication (e.g., a particular dental prosthetic to be used) may be indicated (e.g., may be input by a user), and an appropriate machine learning model may be selected based on the specific tooth number and/or the specific indication.

In an embodiment, the machine learning model may be trained to output an identification of a dental feature as well as separate information indicating one or more of the above (e.g., path of insertion, model orientation, teeth identification, gum identification, excess material identification, etc.). In at least one embodiment, the machine learning model (or a different machine learning model) is trained to perform one or more of: identify teeth represented in height maps, identify gums represented in height maps, identify excess material (e.g., material that is not gums or teeth) in height maps, and/or identify dental features in height maps.

Various embodiments described herein may utilize other methods of producing panoramic 2D images, including the methodologies described in U.S. Patent Application Publication No. 2021/0068773 A1, entitled “Dental Panoramic Views,” which is hereby incorporated by reference herein in its entirety.

FIG. 12B illustrates a flow diagram for a method 1250 of generating projecting segmentation and/or classification information from a panoramic 2D image onto a 3D model of a dental site, in accordance with at least one embodiment. At block 1255, a 3D model of a dental site is generated from an intraoral scan (e.g., by the computing device 105 of the dental office 108 or dental lab 110). In at least one embodiment, the 3D model of the dental site may include a 3D dentition for the lower jaw, the upper jaw, or both, of the patient (e.g., any of the 3D dentitions 210, 660, 730, 760, or 910).

At block 1260, a panoramic 2D image is generated from the 3D model of the dental site, for example, utilizing any of the methods 1000, 1100, or 1200 described in greater detail above.

At block 1265, one or more trained ML models may be utilized to segment/classify dental features identified in the panoramic 2D image. The one or more trained ML models may be trained and utilized in accordance with the methodologies discussed in greater detail above.

At block 1270, information descriptive of the segmentation/classification is projected onto the 3D model of the dental site, for example, by identifying and/or labeling dental features at locations in the 3D model corresponding to those of the panoramic 2D image.

An exemplary process may involve the following operations: (1) generating 2D panoramic images; (2) labeling features of interest in 2D, and mapping the labeled features from 2D to 3D; (3) training a machine learning model on the 3D model with the labeled features.

A further exemplary process may involve the following operations: (1) generating 2D panoramic images; (2) labeling features of interest in the 2D panoramic images; (3) training a machine learning model on the 2D panoramic images with the labeled features; and (4) mapping the results of the machine learning model back to the 3D model.

FIG. 13 illustrates a diagrammatic representation of a machine in the example form of a computing device 1300 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The computing device 1300 may correspond, for example, to computing device 105 and/or computing device 106 of FIG. 1. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein.

The example computing device 1300 includes a processing device 1302, a main memory 1304 (e.g., read-only memory (ROM), flash memory, dynamic random access memory (DRAM) such as synchronous DRAM (SDRAM), etc.), a static memory 1306 (e.g., flash memory, static random access memory (SRAM), etc.), and a secondary memory (e.g., a data storage device 1328), which communicate with each other via a bus 1308.

Processing device 1302 represents one or more general-purpose processors such as a microprocessor, central processing unit, or the like. More particularly, the processing device 1302 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 1302 may also be one or more special-purpose processing devices such as an application specific integrated circuit (ASIC), a field programmable gate array (FPGA), a digital signal processor (DSP), network processor, or the like. Processing device 1302 is configured to execute the processing logic (instructions 1326) for performing operations and steps discussed herein.

The computing device 1300 may further include a network interface device 1322 for communicating with a network 1364. The computing device 1300 also may include a video display unit 1310 (e.g., a liquid crystal display (LCD) or a cathode ray tube (CRT)), an alphanumeric input device 1312 (e.g., a keyboard), a cursor control device 1314 (e.g., a mouse), and a signal generation device 1320 (e.g., a speaker).

The data storage device 1328 may include a machine-readable storage medium (or more specifically a non-transitory computer-readable storage medium) 1324 on which is stored one or more sets of instructions 1326 embodying any one or more of the methodologies or functions described herein, such as instructions for dental modeling logic 116. A non-transitory storage medium refers to a storage medium other than a carrier wave. The instructions 1326 may also reside, completely or at least partially, within the main memory 1304 and/or within the processing device 1302 during execution thereof by the computer device 1300, the main memory 1304 and the processing device 1302 also constituting computer-readable storage media.

The computer-readable storage medium 1324 may also be used to store dental modeling logic 116, which may include one or more machine learning modules, and which may perform the operations described herein above. The computer readable storage medium 1324 may also store a software library containing methods for the dental modeling logic 116. While the computer-readable storage medium 1324 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium other than a carrier wave that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

The following exemplary embodiments are now described:

Embodiment 1: A method comprising: receiving a three-dimensional (3D) model of a dental site generated from one or more intraoral scans; generating a projection target shaped to substantially surround an arch represented by the dental site; computing a surface projection by projecting the 3D model of the dental site onto one or more surfaces of the projection target; and generating at least one panoramic two-dimensional (2D) image of the dental site from the surface projection.

Embodiment 2: The method of Embodiment 1, wherein the surface projection is computed based on a projection path surrounding the arch.

Embodiment 3: The method of Embodiment 2, wherein the at least one panoramic 2D image is generated by orthographic rendering of a flattened mesh generated by projecting the 3D model along the projection path.

Embodiment 4: The method of any of the preceding Embodiments, wherein the projection target comprises a cylindrically-shaped surface that substantially surrounds the arch.

Embodiment 5: The method of any of the preceding Embodiments, wherein the projection target comprises a polynomial curve-shaped surface that substantially surrounds the arch.

Embodiment 6: The method of any of the preceding Embodiments, wherein the projection target comprises: a cylindrically-shaped surface; a first planar surface that extends from a first edge of the cylindrically-shaped surface; and a second planar surface that extends from a second edge of the cylindrically-shaped surface that is opposite the first edge, wherein the cylindrically-shaped surface, the first planar surface, and the second planar surface collectively define a continuous surface that substantially surrounds the arch.

Embodiment 7: The method of Embodiment 6, wherein an angle between the first planar surface and the second planar surface is from about 110° to about 130°.

Embodiment 8: The method of any of the preceding Embodiments, wherein the dental site corresponds to a single jaw, wherein a first panoramic 2D image corresponds to a buccal rendering, and wherein a second panoramic 2D image corresponds to a lingual rendering.

Embodiment 9: The method of Embodiment 8, further comprising: generating for display the buccal rendering, the lingual rendering, and optionally an occlusal rendering of the dental site generated by projecting the 3D model of the dental site onto a flat surface from the occlusal side of the dental site.

Embodiment 10: The method of any of the preceding Embodiments, further comprising: generating for display a panoramic 2D image; labeling a dental feature at a first location in the panoramic 2D image; determining a second location of the 3D model corresponding to the first location of the panoramic 2D image; and assigning a label for the dental feature to the second location of the 3D model, wherein the 3D model is displayable with the label.

Embodiment 11: The method of Embodiment 10, wherein labeling the dental feature comprises one or more of receiving a user input to directly label the dental feature or using a trained machine learning model that has been trained to identify and label dental features in panoramic 2D images.

Embodiment 12: A method comprising: receiving a three-dimensional (3D) model of a dental site generated from one or more intraoral scans; generating a plurality of vertices along an arch represented by the dental site; computing a projection target comprising a plurality of surface segments connected to each other in series at locations of the vertices; scaling the projection target with respect to an arch center located within a central region of the arch such that the projection target substantially surrounds the arch; computing a surface projection by projecting the 3D model of the dental site onto each of the surface segments of the projection target; and generating at least one panoramic two-dimensional (2D) image of the dental site from the surface projection.

Embodiment 13: The method of Embodiment 12, wherein one or more of the plurality of vertices is positioned at a tooth center, and wherein the number of vertices is greater than 5.

Embodiment 14: A method comprising: receiving a three-dimensional (3D) model of a dental site generated from one or more intraoral scans; generating a projection target shaped to substantially surround an arch represented by the dental site; computing a first surface projection by projecting the 3D model of the dental site onto one or more surfaces of the projection target along a buccal direction; computing a second surface projection by projecting the 3D model of the dental site onto one or more surfaces of the projection target along a lingual direction; and generating at least one panoramic two-dimensional (2D) image by combining the first surface projection and the second surface projection.

Embodiment 15: The method of Embodiment 14, wherein generating the at least one panoramic 2D image comprises marking regions of a panoramic 2D image corresponding to overlapping regions of the 3D model identified from the first and second surface projections.

Embodiment 16: An intraoral scanning system comprising: an intraoral scanner; and a computing device operatively connected to the intraoral scanner, wherein the computing device is to perform the method of any of Embodiments 1-15 responsive to generating the one or more intraoral scans using the intraoral scanner.

Embodiment 17: A non-transitory computer readable medium comprising instructions that, when executed by a processing device, cause the processing device to perform the method of any of Embodiments 1-15.

Embodiment 18: A system comprising: a memory; and a processing device to execute instructions from the memory to perform a method comprising: receiving a three-dimensional (3D) model of a dental site generated from one or more intraoral scans; generating a projection target shaped to substantially surround an arch represented by the dental site; computing a surface projection by projecting the 3D model of the dental site onto one or more surfaces of the projection target; and generating at least one panoramic two-dimensional (2D) image of the dental site from the surface projection.

Embodiment 19: The system of Embodiment 18, wherein the surface projection is computed based on a projection path surrounding the arch, and wherein the at least one panoramic 2D image is generated by orthographic rendering of a flattened mesh generated by projecting the 3D model along the projection path.

Embodiment 20: The system of either Embodiment 18 or Embodiment 19, wherein the projection target comprises a cylindrically-shaped surface that substantially surrounds the arch.

Embodiment 21: The system of any of Embodiments 18-20, wherein the projection target comprises a polynomial curve-shaped surface that substantially surrounds the arch.

Embodiment 22: The system of any of Embodiments 18-21, wherein the projection target comprises: a cylindrically-shaped surface; a first planar surface that extends from a first edge of the cylindrically-shaped surface; and a second planar surface that extends from a second edge of the cylindrically-shaped surface that is opposite the first edge, wherein the cylindrically-shaped surface, the first planar surface, and the second planar surface collectively define a continuous surface that substantially surrounds the arch, and wherein an angle between the first planar surface and the second planar surface is from about 110° to about 130°.

Embodiment 23: The system of any of Embodiments 18-22, wherein the dental site corresponds to a single jaw, wherein a first panoramic 2D image corresponds to a buccal rendering, and wherein a second panoramic 2D image corresponds to a lingual rendering, and wherein the method further comprises: generating for display the buccal rendering, the lingual rendering, and optionally an occlusal rendering of the dental site generated by projecting the 3D model of the dental site onto a flat surface from the occlusal side of the dental site.

Embodiment 24: The system of any of Embodiments 18-22, further comprising: generating for display a panoramic 2D image; labeling a dental feature at a first location in the panoramic 2D image; determining a second location of the 3D model corresponding to the first location of the panoramic 2D image; and assigning a label for the dental feature to the second location of the 3D model, wherein the 3D model is displayable with the label.

Embodiment 25: The system of Embodiment 24, wherein labeling the dental feature comprises one or more of receiving a user input to directly label the dental feature or using a trained machine learning model that has been trained to identify and label dental features in panoramic 2D images.

While the present disclosure is described with respect to the specific application of dental evaluation for humans, the present disclosure is not limited thereto. The techniques described herein can equally be applied to any other medical applications. For example, techniques described can be utilized for imaging generally, and in particular for imaging and characterizing elements of human or animal anatomy such as eyes, nose, other facial elements, bone structures, etc.

Claim language or other language herein reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

It is to be understood that the above description is intended to be illustrative, and not restrictive. Many other embodiments will be apparent upon reading and understanding the above description. Although embodiments of the present disclosure have been described with reference to specific example embodiments, it will be recognized that the disclosure is not limited to the embodiments described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

What is claimed is:

1. A method comprising:
receiving a three-dimensional (3D) model of a dental site generated from one or more intraoral scans;
generating a projection target shaped to substantially surround an arch represented by the dental site;
computing a surface projection by projecting the 3D model of the dental site onto one or more surfaces of the projection target; and
generating at least one panoramic two-dimensional (2D) image of the dental site from the surface projection.

2. The method of claim 1, wherein the surface projection is computed based on a projection path surrounding the arch.

3. The method of claim 2, wherein the at least one panoramic 2D image is generated by orthographic rendering of a flattened mesh generated by projecting the 3D model along the projection path.

4. The method of claim 1, wherein the projection target comprises a cylindrically-shaped surface that substantially surrounds the arch.

5. The method of claim 1, wherein the projection target comprises a polynomial curve-shaped surface that substantially surrounds the arch.

6. The method of claim 1, wherein the projection target comprises:
a cylindrically-shaped surface;
a first planar surface that extends from a first edge of the cylindrically-shaped surface; and
a second planar surface that extends from a second edge of the cylindrically-shaped surface that is opposite the first edge,
wherein the cylindrically-shaped surface, the first planar surface, and the second planar surface collectively define a continuous surface that substantially surrounds the arch.

7. The method of claim 6, wherein an angle between the first planar surface and the second planar surface is from about 110° to about 130°.

8. The method of claim 1, wherein the dental site corresponds to a single jaw, wherein a first panoramic 2D image corresponds to a buccal rendering, and wherein a second panoramic 2D image corresponds to a lingual rendering.

9. The method of claim 8, further comprising:
generating for display the buccal rendering, the lingual rendering, and optionally an occlusal rendering of the dental site generated by projecting the 3D model of the dental site onto a flat surface from the occlusal side of the dental site.

10. The method of claim 1, further comprising:
generating for display a panoramic 2D image;
labeling a dental feature at a first location in the panoramic 2D image;
determining a second location of the 3D model corresponding to the first location of the panoramic 2D image; and
assigning a label for the dental feature to the second location of the 3D model, wherein the 3D model is displayable with the label.

11. The method of claim 10, wherein labeling the dental feature comprises one or more of receiving a user input to directly label the dental feature or using a trained machine learning model that has been trained to identify and label dental features in panoramic 2D images.

12. A method comprising:
receiving a three-dimensional (3D) model of a dental site generated from one or more intraoral scans;
generating a plurality of vertices along an arch represented by the dental site;
computing a projection target comprising a plurality of surface segments connected to each other in series at locations of the vertices;
scaling the projection target with respect to an arch center located within a central region of the arch such that the projection target substantially surrounds the arch;
computing a surface projection by projecting the 3D model of the dental site onto each of the surface segments of the projection target; and
generating at least one panoramic two-dimensional (2D) image of the dental site from the surface projection.

13. The method of claim 12, wherein one or more of the plurality of vertices is positioned at a tooth center, and wherein the number of vertices is greater than 5.

14. A method comprising:
receiving a three-dimensional (3D) model of a dental site generated from one or more intraoral scans;
generating a projection target shaped to substantially surround an arch represented by the dental site;
computing a first surface projection by projecting the 3D model of the dental site onto one or more surfaces of the projection target along a buccal direction;
computing a second surface projection by projecting the 3D model of the dental site onto one or more surfaces of the projection target along a lingual direction; and
generating at least one panoramic two-dimensional (2D) image by combining the first surface projection and the second surface projection.

15. The method of claim 14, wherein generating the at least one panoramic 2D image comprises marking regions of a panoramic 2D image corresponding to overlapping regions of the 3D model identified from the first and second surface projections.

16. An intraoral scanning system comprising:
an intraoral scanner; and
a computing device operatively connected to the intraoral scanner, wherein the computing device is to perform the method of claim 1 responsive to generating the one or more intraoral scans using the intraoral scanner.

17. A non-transitory computer readable medium comprising instructions that, when executed by a processing device, cause the processing device to perform the method of claim 1.

18. A system comprising:
a memory; and
a processing device to execute instructions from the memory to perform a method comprising:
receiving a three-dimensional (3D) model of a dental site generated from one or more intraoral scans;
generating a projection target shaped to substantially surround an arch represented by the dental site;
computing a surface projection by projecting the 3D model of the dental site onto one or more surfaces of the projection target; and
generating at least one panoramic two-dimensional (2D) image of the dental site from the surface projection.

19. The system of claim 18, wherein the surface projection is computed based on a projection path surrounding the arch, and wherein the at least one panoramic 2D image is generated by orthographic rendering of a flattened mesh generated by projecting the 3D model along the projection path.

20. The system of claim 18, wherein the projection target comprises a cylindrically-shaped surface that substantially surrounds the arch.

21. The system of claim 18, wherein the projection target comprises a polynomial curve-shaped surface that substantially surrounds the arch.

22. The system of claim 18, wherein the projection target comprises:
a cylindrically-shaped surface;
a first planar surface that extends from a first edge of the cylindrically-shaped surface; and
a second planar surface that extends from a second edge of the cylindrically-shaped surface that is opposite the first edge,
wherein the cylindrically-shaped surface, the first planar surface, and the second planar surface collectively define a continuous surface that substantially surrounds the arch, and wherein an angle between the first planar surface and the second planar surface is from about 110° to about 130°.

23. The system of claim 18, wherein the dental site corresponds to a single jaw, wherein a first panoramic 2D image corresponds to a buccal rendering, and wherein a second panoramic 2D image corresponds to a lingual rendering, and wherein the method further comprises:
generating for display the buccal rendering, the lingual rendering, and optionally an occlusal rendering of the dental site generated by projecting the 3D model of the dental site onto a flat surface from the occlusal side of the dental site.

24. The system of claim 18, further comprising:
generating for display a panoramic 2D image;
labeling a dental feature at a first location in the panoramic 2D image;
determining a second location of the 3D model corresponding to the first location of the panoramic 2D image; and
assigning a label for the dental feature to the second location of the 3D model, wherein the 3D model is displayable with the label.

25. The system of claim 24, wherein labeling the dental feature comprises one or more of receiving a user input to directly label the dental feature or using a trained machine learning model that has been trained to identify and label dental features in panoramic 2D images.
